(19) United States
(12) Patent Application Publication
(10) Pub. No.: US 2004/0003429 A1
(43) Pub. Date: Jan. 1, 2004

(54) Pest control system

(75) Inventors: Roger Craig, Cheshire (GB); Charalambos Savakis, Crete (GR)

Correspondence Address: PALMER & DODGE, LLP, KATHLEEN M. WILLIAMS, 111 HUNTINGTON AVENUE, BOSTON, MA, 02199, US

(73) Assignee: Minos Biosystems Limited

(21) Appl. No.: 10/385,768

(22) Filed: Mar. 11, 2003

Related U.S. Application Data
(63) Continuation of application No. PCT/GB01/04065, filed on Sep. 11, 2001.
(60) Provisional application No. 60/232,366, filed on Sep. 14, 2000.

(30) Foreign Application Priority Data
Sep. 11, 2000 (GB) 0022193.7

(51) Int. Cl. A01H 1/00; C12N 15/82; A01N 57/00; A01N 43/40

(52) U.S. Cl. 800/279; 514/114; 514/343; 514/229.2

(57) ABSTRACT

The present invention provides a method for controlling a population of target pests, comprising: a) providing gene comprising a coding sequence encoding one constituent of an enzyme/pro-pesticide system and a promoter capable of driving the coding sequence in the target plants or vertebrate host; b) transforming the target plant or vertebrate with the gene; and c) administering to the transformed population of target plant or vertebrates the remaining constituents of the enzyme/pro-pesticide system such that inactive pro-pesticide is converted to active pesticide on or within the transformed plant or vertebrate host leading to the death of any invading population of target pests.

### FIELD OF INVENTION

The present invention relates to methods for controlling pest populations using genetic techniques.

### BACKGROUND OF THE INVENTION

Insects fungi, nematodes, protozoa, bacteria and viruses are responsible for widespread damage to crops and animals world-wide with enormous concomitant economic consequences. To try to reduce pest-inflicted damage, resources have been devoted to the development and deployment of pesticide, which control pest populations by killing target pests. Although pesticide are in many cases effective, they are known to be toxic to life forms other than target insects, which has important environmental consequences. It would therefore be advantageous to develop inactive pro-pesticides which are converted into their active form predominantly in the cells and tissues of plants and animals on which the pests feed and replicate.

Attempts have been made to control pests in particular insects by biological means. For example, methods currently employed to control the populations of certain members of the dipteran class include the release of sterile males.

For example, as set forth in U.S. Pat. No. 5,840,865, the Mediterranean fruit-fly (Medfly) Ceratitis capitata is a major agricultural pest for many fruit species that is geographically widespread in tropical and temperate regions. The Medfly has been introduced relatively recently into the New World, and appears to be spreading rapidly, threatening fruit producing areas in North America (Carey, J. R., Science 253: 1369 (1991)). Since the mid 1970's, the sterile insect technique has been used for Medfly eradication and control. This method relies on the decrease in or collapse of fly populations following releases of large numbers of sterile insects over infested areas, and offers an environmentally attractive alternative to massive spraying with insecticides (Knipling, E. F., Science 130: 902 (1959)).

Although the use of sterile male insects slows Medfly population growth and may lead to its temporary collapse, it does not lead to destruction of female insects, which are responsible for crop damage. Moreover, since the sterile males do not reproduce, the method requires repeated releases of sterile males into the environment.

There therefore remains a need for a control technique for Medfly and other insects and other pests which can selectively destroy target pests but which is environmentally more acceptable than the mass spraying of toxic pesticides.

Furthermore, many human and veterinary health issues are associated with the spread of disease by insects and other pests. Examples include mosquitoes, tse-tse flies and the common housefly. Control of pest populations which endanger human or animal health is thus also desirable.

### SUMMARY OF THE INVENTION

The present invention provides a method for controlling a population of target pests, comprising:

a) providing a gene comprising a coding sequence encoding one constituent of an enzyme/pro-pesticide system and a promoter capable of driving the coding sequence in the target plant or vertebrate host;

b) transforming the population of target plants or vertebrates with the gene; and

c) administering to the transformed population of target plants or vertebrates the remaining constituent(s) of the enzyme/pro-pesticide system, such that inactive pro-pesticide is converted to active pesticide within the transformed plant, or vertebrate host leading to the death of any invading population of target pests.

As used herein, a “population of target pests” refers to a group of insects and other parasites and infectious organism including fungi, acancides protrozoa, bacteria, and viruses which invade the host plant or vertebrate to feed or replicate whether delimited along species or geographical lines, or both, which it is desired to be controlled. For example, a population of pests may refer to a given species of pest which infests a particular crop or vertebrate in a given geographical area. Alternatively, it may refer to all pests infesting any crop or vertebrate in a geographical area, or a given species without reference to any geographical limitation, or a population of pests which is responsible for a human or veterinary health problem, such as the spread of malaria. Target pests are the individual members of the population of pests.

“Plant or vertebrate host” as used herein means any host organism to be protected from pests through incorporation of an enzyme/pro-drug system. This encompasses incorporation of an enzyme/pro-drug system into symbiotic organisms which live within the target host.

“Pesticide” as used herein means any pesticide that is meant to control any target pest by targeting the pest cells. Insecticides which can be modified according to the invention to be pro-insecticides include but are not limited to imidacloprid and methamidophos.

“Pro-insecticide”, “pro drug” and “pro-pesticide” are used interchangeably and as used herein mean any substantially inactive or substantially non-toxic substance in the absence of a converting enzyme, or mixture comprising such substance that can be converted to active or toxic substance by the action of an enzyme. Pro-pesticides can be specifically designed for the purpose or preferably designed by chemically modifying existing pesticides using, for example, amidation methylation or acetylation as taught herein. The term “substantially” as used herein means “pro-insecticide”, “pro-drug” and “pro-pesticide”, which is at least 50%; 60%; 70%; 80; 90%; 95%; 98% and up to and including 100% inactive when compared to the active form.

“Enzyme” as used herein means a reaction catalysing substance including but not limited to RNA, protein or polypeptide or a fragment of such protein or polypeptide. An “enzyme” as used herein catalyses a reaction which converts a pro-insecticide or pro-pesticide to be an insecticide or pesticide, respectively. Examples of enzymes include but are not limited to oxidases, esterases, and amidases, or proteases.

“Control” as used herein refers to the limitation, prevention or reduction of growth, i.e., by at least about 10% per generation, preferably at least about 50%, 80%, or even up to and including 100% of the insect population. Preferably, this is achieved by killing target pests. Advantageously, the population of pests is eliminated.

“Gene”, as used herein, refers to a nucleic acid sequence, usually DNA, which encodes a polypeptide or protein and additionally comprises the nucleic acid sequences required to transcribe the coding sequence in a suitable host cell. The nucleotide sequence encoding the polypeptide or protein is referred to herein as a “coding sequence” and the sequences required for regulation of sequence transcription are referred to as “control sequence”, such as “enhance” “promoter” or “locus contact regimen”.

The coding sequence encodes one constituent of an enzyme/pro-pesticide system. The constituent may be any one or more parts of the system, as long as it is not itself sufficient to produce or transform itself into the active pesticide from the pro-pesticide. Thus, the constituent is preferably an enzyme or a fragment of an enzyme which is responsible for pro-pesticide activation. Alternatively, it may be the pro-pesticide itself. The remaining constituent(s) of the enzyme/pro-pesticide system are administered separately, for example by spraying, thus killing the target pests which invade the host plant or vertebrate which express the coding sequence according to the invention.

A feature of the present invention is that the promoter used to drive transcription of the coding sequence is functionally active in the host plant or vertebrate, the target of pest activity. This means that the coding sequence is expressed substantially only in the host plant or vertebrate.

Thus the application of relatively non-toxic pro-pesticides will be converted to the toxic pesticide within the transformed host plant or vertebrate, thereby protecting other species in the environment from the effects of mass applications of a toxic pesticide.

In a further aspect, the invention provides a vector which is capable of transforming a target plant or vertebrate host cell, which vector comprises a gene comprising a coding sequence encoding one constituent of an enzyme/pro-drug system and a promoter capable of driving the coding sequence in target plant or vertebrate hosts.

### BRIEF DESCRIPTION OF THE FIGURES

FIG. 1. Effect of acephate (ace) and methamidophos (met) on Drosophila melanogaster (strain OR/R) and Ceratitis capitata (strain Benakeion) expressed as (%) survival 16 h after insecticide-treatment at indicated concentrations.

FIG. 2. PCR amplification of Drosophila amidase genes. Lanes: 1,No DNA control; 2,CG5112 primers; 3, CG5191; 4, CG7900; 5, CG7910; 6, CG8839; 7, size markers

FIG. 3. Southern analysis of amidase-related genes in various flies. Lanes: 1, Musca domestica (house fly); 2, Bactrocera oleae (olive fly); 3, Cetatitis capitata (medfly); 4, Drosophila melanogaster (vinegar fly).

### DETAILED DESCRIPTION OF THE INVENTION

The present invention is applicable to control using pro-pesticides of any homogeneous or heterogeneous target pest population which can invade or infect a plant or vertebrate host transformed with a gene encoding an enzyme which will convert the pro-pesticide to its active toxic form.

For example the phosphoramidate insecticide methamidophos has broad spectrum activity against aphids, caterpillars, and mites, but has high mammalian toxicity. This contrasts to an inactive analogue acephate which has low mammalian toxicity and can be converted to the toxic methamidophos by amidase enzymes present in certain but not all plants and insects (see Magee in Insecticide Mode of Action, 1982, Academic Press, 101-161).

Thus the present invention teaches that transgenic plants and vertebrates of economical importance can be generated which express proinsecticide converting enzymes in their cells and tissues capable of converting inactive and non-toxic pro-pesticides into their active form in the tissues and cells of the host species. The localised presence of the active pesticide will eliminate pests such as insects, nematodes, protozoa, fungi, bacteria and viruses feeding on or replicating in the host plant or vertebrate in the absence of indiscriminate environmental effects seen by the wide scale application of the toxic pesticide alone. In the preferred embodiment, the present invention is used for the control of insect populations of agricultural importance especially for the control of sucking, chewing and biting insects such as rice hopper, aphids, thrips, whiteflies, termites, turf insects and soil insects which attack rice, cereals, maize, potatoes, vegetables, sugar beet, soft fruit, citrus fruit, olives, cotton, hops, vines, tobacco and turf.

Vectors and Transformation According to the Invention

Vectors for use in tissue-specific targeting of genes in transgenic plants will typically include tissue specific promoters and may also include other tissue-specific control elements such as enhancer sequences. Promoters which direct specific or enhanced expression in certain plant tissues include, for example, the rbcS promoter, specific for green tissue, the ocs, nos and mas promoters which have higher activity in roots or wounded leaf tissue: a truncated (˜90 to 18) 35S promoter which directs enhanced expression in roots, an Δ-tubulin gene that directs expression in roots and promoters derived from zein storage protein genes which direct expression in endosperm (See Taque et al. (1988) Plant Physiology 86:506). It is particularly contemplated that one may advantageously use the 16 bp ocs enhancer element from the octopine synthase (ocs) gene (Ellis et al., 1987: Bonchez et al., 1989), especially when present in multiple copies, to achieve enhanced expression in roots.

The transgenic plant can be dicotyledonous (a dicot) or monocotylodonous (a moncot). Examples of monocot plants are grasses, such as meadow grass (blue grass, Poa), forage grass such as festoca, lolium, temperate grass, such as Agrostis, and cereals, e.g., ,wheat, oats, rye, barley, rice, sorghum, and maize (corn). Examples of dicot plants are tobacco, legumes, such as lupins, potato, sugar beet, pea, bean and soybean and crociferdus plants (family Brassicacrae), such as cauliflower, rape seed and the closely related model organism Arabidopsis thaliana. The transgenic plants may also be soft and citrus fruit trees.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, BioTechnology 8: 535; Shimamoto et al., 1989, Nature 338: 274). Presently, Agrobacterium tumefaciens-mediated gene transfer is the method of choice for generating transgenic dicots (for a review, see Hooykas and Schilperoort 1992, Plant Molecular Biology 19: 15-38). Presently, the method of choice for generating transgenic monocots is particle bombardment (microscopic gold or tungsten particles coated with the transforming DNA) of embryonic calli or developing embryos (Christon, 1992, Plant Journal 2: 275-281; Shimamoto, 1994, Current Opinion Biotechnology 5: 158-162).

Production of Transgenic Animals

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Advances in technologies for embryo micromanipulation now permit introduction of heterologous DNA into, for example, fertilised mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals. Those techniques as well known. See reviews of standard laboratory procedures for microinjection of heterologous DNAs into mammalian fertlised ova, including Hogan et al., Manipulating the Mouse Embryo, (Cold Spring Harbor Press 1986); Krimpenfort et al., Bio/Technology 9:844 (1991); Palmiter et al., Cell, 41: 343 (1985); Kraemer et al., Genetic manipulation of the Mammalian Embryo, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated herein by reference

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology as described in Schnieke, A. E. et al., 1997, Science, 278: 2130 and Cibelli, J. B. et al., 1998, Science, 280: 1256. Using this method, fibroblasts from donor animals are stably transfected with a plasmid incorporating the coding sequences for a binding domain or binding partner of interest under the control of regulatory. Stable transfectants are then fused to enucleated oocytes, cultured and transferred into female recipients.

Analysis of animals which may contain transgenic sequences would typically be performed by either PCR or Southern blot analysis following standard methods.

By way of a specific example for the construction of transgenic mammals, such as cows, nucleotide constructs comprising a proinsecticide converting enzyme are microinjected using, for example, the technique described in U.S. Pat No. 4,873,191, into oocytes which are obtained from ovaries freshly removed from the mammal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronised in the intended recipient mammals, such as cattle, by administering coprostanol. Oestrous is produced within two days and the embryos are transferred to the recipients 5-7 days after oestrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric stains which exclusively comprise the ES progeny can be obtained using conventional cross-breeding. This technique is described, for example, in WO91/10741.

Pro-Drug Systems Useful According to the Present Invention

Pro-drug-converting enzyme genes, also known as “suicide” genes have been used to subsequently activate cytotoxic drugs selectively in transfected tumour cells. Enzyme/pro-drug systems are known in the art. A pro-drug is a drug, often a potentially toxic drug, which is selectively activated, i.e. rendered toxic, by the action of an enzyme. Enzyme/pro drug systems rely on the delivery of an enzyme to target cells or organisms, before administration of the prodrug. Only target cells or organisms which express the enzyme will be affected by the pro-drug. One common enzyme/pro-drug system is the 5-fluorouracil/cytosine deaminase system, in which the non-toxic precursor 5-fluorocytosine (5-FC) is converted to the cytotoxic drug 5-fluorouracil (5-FU) by the action of cytosine deaminase (see Austin & Huber, (1993) Mol. Pharmacol. 43:380-387).

In one embodiment, enzymes that can be used for converting a pro-pesticide into an active pesticide, such as esterases, amidases and mixed-function oxidases which are also called P450, can be characterised and cloned from a variety of organisms, including eubacteria, archaebacteria and eukaria. A typical strategy for utilising such an enzyme, e.g. metabolising a specific pro-pesticide, would entail screening of organisms for presence of the enzymatic activity. A collection of organisms can be used for this purpose, such as a collection of cultured soil bacteria, a collection of insect tissues or cells or a collection of plant tissues or cells.

Screening can be performed, for example, by incubating the pro-pesticide with an extract from tissues or from bacteria. In the case of mixed function oxidases that are localised in microsomes from eukaryotic tissues, a microsomal fraction can be used as source of the activity. A microsomal fraction can be isolated by routine subcellular fractionation procedures. High speed supernatants of cell extracts can be used for other soluble enzymes. Determination of enzymatic activity can be performed by detecting the reaction product(s) by standard chemical analytical technologies well know to one skilled in art. For example, reaction product(s) can be separated using gas chromatography (GC). Mass spectrometry coupled to GC (GCMS) can be used to confirm the structure of the compounds.

Once the enzyme activity is determined, the gene encoding the activating enzyme can be cloned by using a variety of different cloning methods well know in art. For example, a nucleic acid library of the organism is generated using standard recombinant DNA technology. A genomic library for bacterial genes or a cDNA library for eukaryote genes can then be screened using standard methodology.

In one embodiment, hybridisation with nucleic acid probes from a related gene, e.g. a cloned esterase or P450 gene from an evolutionarily related organism, is used. Alternatively, screening can be done by complementation. In the latter approach, an expression library, i.e. a library construed in a commercially available expression plasmid or viral vector, is used to transfect cells that do not normally express the enzymatic activity, and the clone containing the activating gene is isolated by screening individual colonies of the library for expression of enzymatic activity. A newly identified gene can then be cloned into vectors useful according to the invention, describe below.

Pro-drugs, as well as being inactive in organisms or cells that cannot convert them, may also have other advantages, such as improved lipid solubility and/or chemical stability.

Pro-insecticides or pro-drug toxins that have been designed to be convertible only in certain insects, are known in the art and have been prepared, for example, by selective derivatisation of the final toxin, especially in the case of organophosphates and carbamates. Examples include precursors of acetylcholinesterase inhibitors, such as parathion and profenofos, which are oxidised into active insecticides by enzymes of the cytochrome PA450 family. However, application of proinsecticides to insect populations only kills the insects which naturally express the enzyme. To render insect pests which do not naturally express the converting enzyme susceptible requires transfer of the gene encoding the converting enzyme into the host and a means of transfer of the gene in an environmentally acceptable manner within the target insect population.

In one embodiment, the converting enzyme coding sequence encodes the esterase enzyme which converts DPX-JW062 into its active metabolite as disclosed by Wing et al. incorporated herein by reference (Wing et al., Arch insect Biochem Physiol 37:91-103, 1998). The pro-insecticide is, in this aspect of the invention, DPX-JW062. DPX-JW062 is an oxadiazine compound bioconverted in lepdoptera into a potent toxin which blocks voltage-gated sodium channels. DPX-JW062 has low activity/toxicity in other organisms. Consequently, the expression of the lepdoptera converting enzyme in plants or vertebrate will make invading insects susceptible to DPX-JW062 induced toxicity, whether or not the target cited contains an enzyme capable of converting DPX-JW062 to its toxic form.

Alternatively, the coding sequence encodes an enzyme of the cytochrome P450 family. Although most insects possess cytochrome P450 family enzymes, the efficiency of bioconversion of proinsecticides converted by such enzymes may be increased by transforming the insect with a more efficient and/or overexpressed enzyme, allowing a decrease in the effective dose of the proinsecticide. Suitable proinsecticides in this aspect of the invention include organophosphates and carbamates. For example, the proinsecticide may be an acetylcholinesterase inhibitor such as parathion and profenofos. The cloning and expression of the aphid cytochrome P450 enzymes responsible for the conversion of parathion and profenofos in plants or vertebrates to them and we found would allow a broadening of the susceptible range of invading insects, whether or not the insect enzyme the appropriate converting enzyme.

Generally, genes useful in the practice of the invention, as well as vectors encoding them, may be prepared according to standard approaches used in molecular biology. See, for example, Sambrook et al., Molecular Cloning, A Laboratory Manual (1989) and Ausubel et al., Short Protocols in Molecular Biology (1999) 4th Ed, John Wiley & Sons, Inc.

Designing Pro-Pesticides from Pre-Existing Pesticides According to the Invention

To create non-toxic pro-insecticides, currently available insecticides can be rendered inactive by derivatising a chemical group that is required for biological activity. Such groups include, but are not limited to, amino or imino groups. Derivatisation can be performed by, for example, methylation or acetylation.

Metabolic activation is an enzymatic process in the target species that converts the pesticide to a biologically more active structure. Thus, weak inhibitors or noninhibitors may be accidentally changed into products of lethal toxicity by systems designed to degrade xenobiotic compounds (p. 129 in Insecticide Mode of Action ed. Joel R. Coats, Academic Press, 1982).

In the general field of phosphate insecticides, the most common activation process mediated by mixed-function oxidase is the P→S to P→O conversion, e.g. parathion to paraoxon. (Nakatsogawa and Morelli, 1976; Ero, 1974; Fukuto, 1978).

In one embodiment of the invention, the acephate-methamidophos propesticide-pesticide system can be used. Methamidophos (O, S-dimethyl phosphoramidothicate, C₂H₈NO₂PS) was introduced in 1969 by Chevron Chemical and Bayer. It is a highly active, systemic, residual organophosphate insecticide/acaricide with contact and stomach action. Methamidophos is a potent acetylcholinesterase (AchE) inhibitor. It is effective against chewing and sucking insects and is used to control aphids, flea beetles, worms, whiteflies, thrips, cabbage loopers, Colorado potato beetles, potato tubeworms, armyworms, mites, leafhoppers, and many others. Toxicity of methamidophos has been determined as acute oral LD50 values of 21 and 16 mg/kg body weight for male and female rats respectively.

Acephate is a N-acetyl derivative of methamidophos (O, S dimethyl acetylphosphoramidothicate, C₉H₁₀NO₃PS) which was introduced by Chevron Chemical as a second-generation improvement of methamidophos. It is used for control of a wide range of biting and sucking insects, especially aphids, including resistant species, in fruit, vegetables (e.g. potatoes and sugar beets), vine, and hop cultivation and in horticulture (e.g. on roses and chrysanthemums grown outdoors). Acephate and its primary metabolite, methamidophos, are toxic to Heliothis spp. that are considered resistant to other organophosphate insecticides. Toxicity of Acephate has been determined as acute oral LD50 Value A 945 and 866 mg/kg for male and female rats respectively (45 to 54-fold less toxic than its metabolite methamidophos).

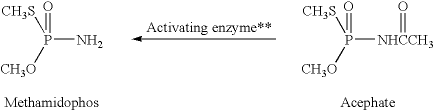

Acephate itself is not an AchE inhibitor; its conversion to methamidophos, which is an inhibitor, requires an enzyme-mediated cleavage of the carbonyl-N bond. Nearly all living systems contain amidases that cleave simple aliphatic amides. However, acephate is not a simple amide and some nonspecific amidases may find it a poor substrate. Methamidophos has been observed as the principle metabolite of acephate in bean, cabbage and tomato seedlings (Tucker, 1972, Report, Chevron Chemical Company, Richmont, Calif.). The cleavage of the carbonyl-N bond is not a nonspecific process, as is indicated by findings that activity of the activating amidases is highly sensitive to variations in the acephate structure.

There appears to be a direct relation of the activation reaction and toxicity in target insects. In the tobacco budworm Heliothis virescens acephate and methamidophos show comparable activity. A small amount of methamidophos was observed in the budworm larvae within 2 hours of a topical acephate application. However, in the adult boll weevil Anthonomus granids Boheman methamidophos is 75-fold more toxic than acephate despite rapid absorbsion of both compounds. No metabolism of acephate to methamidophos was observed in the boll weevil, a fact consistent with its low toxicity (Bull, 1979, J. Agric. Food Chem. 27:268).

Many living organisms possess one or more amidases that cleave a significant fraction of applied acephate to methamidophos. Animals such as the rat (Tucker, 1976) and the female white mouse (Kao and Fukuto, 1977) also convert a significant amount of acephate or its analogues to methamidophos. In their study on the propionyl and hexanoyl analogues, Kao and Fukuto were able to relate the toxicity directly to the amount of activation. This strongly suggests that unconverted acephate is essentially nontoxic.

Thus the combination of a gene encoding an amidase enzyme capable of converting acephate to its active form methamidophos will render an insect susceptible to applications of the previously non-toxic acephate.

It has also been reported that the toxicity of acephate analogues to the house fly is consistent with the amount of metabolically formed methamidophos; the propionyl analogue generates substantially more methamidophos in this species and is 35-fold more toxic (Kao and Fukato, 1977, Pestc. Biochem. Physiol. 7:83).

In another embodiment of the invention, Imidacloprid (1-[8-chloro-3-pyridinyl)methyl]-N-nitro-2-imidazolidinimine, C₉H₁₀CIN₅O₂) and N-Me-imidacloprid can be used as a pro-pesticide system.

Imidacloprid is a systemic, chloro-nicotinyl insecticide for the control of sucking insects including rice hoppers, aphids, thrips, whiteflies, termites, turf insects, soil insects and some beetles. It is most commonly used on rice, cereal, maize, potatoes, vegetables, sugar beets, fruit, cotton, hops and turf, and is especially systemic when used as a seed or soil treatment. The chemical works by interfering with the nicotinergic acetylcholine receptor (nAcR) which is more abundant in insects than in warm-blooded animals. It is effective on contact and via stomach action. (Kidd, H. and James, D. R., Eds. The Agrochemicals Handbook, Third Edition. Royal Society of Chemistry Information Services, Cambridge, UK, 1991 (As Updated). 10-2). Toxicity for imidacloprid has been determined as acute oral LD50 in rats is 450 mg/kg body weight.

N-Me-imidacloprid is an imidacloprid pro-insecticide. Introduction of a methyl group at the 3 position of the imidazole ring imidacloprid decreases binding affinity to nAcR 100-fold. However, N-Me-imidacloprid is as potent as imidacloprid in the housefly. N-Me-imidacloprid can be converted to imidacloprid by an in vitro mixed function oxidase system (Yamamoto et al., 1998, Arch. Insect Biochem. Physiol. 37:24). Currently, no toxicity data are available for N-Me-imidacloprid.

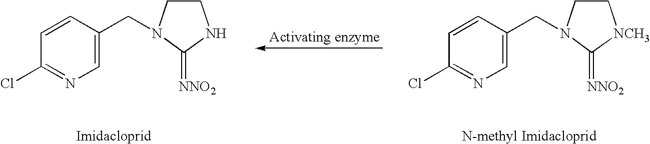

The conversion of N-Me-imidacloprid to imidacloprid in the housefly indicates the presence of enzymes capable of effecting pro-pesticide conversion. See Magee (1982) in Structure-Act In Vivo relationships in phosphoramidates, Chp 5, Insecticide mode a action Academic Press.

The resulting derivative (pro-pesticide) can be metabolised to the active pesticide by enzymes or “activating enzymes” that occur or are transfected into target insects. Several activating enzymes can be used including but not limited to oxidases (P450), esterases and amidases.

In summary a gene encoding an activating enzyme that converts a non-toxic pro-pesticide into an active pesticide is introduced into the genome of the host plant or vertebrate species that do not normally produce the activating enzyme or have low levels of activating enzyme. Pests which invade the transformed pest plants or vertebrate can then be selectively eliminated after administration of a non-toxic pro-pesticide subsequently converted to the active form in the host species.

Designing a pro-pesticide which is based on a registered pesticide such as imidacloprid has an important economical advantage over introducing a novel pro-pesticide: The known toxicology of the pesticide is utilised instead of establishing the extensive toxicology analysis of a novel compound which decreases time to market and costs for toxicology analysis.

The invention is further illustrated in the following examples, which are non-limiting.

### EXAMPLES

### Genetically Enhanced Plants Sensitised to Pro-Pesticides

A number of pesticides are used in the form of pro-pesticides, i.e. biologically inactive (or low-activity) derivative compounds that are converted to the active form through the action of enzymes in the target organism. Such pro-pesticides are generally safer than the corresponding active compounds because of their lower toxicity in organisms that do not produce (or underproduce) the activating enzyme. They can also offer other advantages, such as increased stability or increased penetration into organisms or cells. A method to utilise such pro-pesticides for targeted population control of harmful insects has been disclosed in International Patent Application WO0110220 (“Insect Control System”). In this method, called Sensitisation of Insect Populations to Pro-insecticides (SIPP), a gene that activates a pro-insecticide is introduced into the genome of the target species and recombinant insects are released in the appropriate areas under conditions that can drive the gene into the target populations. SIPP is possible only for insects species for which there is mass rearing technology.

An alternative to SIPP is a method based on production of pro-pesticide activating enzymes (PAE's) in plants. In this approach, transgenic target plants are produced that express a PAE in the appropriate tissues (e.g. leaves or roots). Application of the appropriate pro-pesticide will lead to control of insects which feed on the tissues of the plant

### Example 1

### The Acephate-Methamidophos System

Methamidophos is a widely used, potent organophosphate which exhibits high toxicity for insects and mammals. Acephate is a much less toxic pro-insecticide derivative, which is converted to Methamidophos by the action of amidases present in a variety of insects. The example describes cloning of an amidase-encoding gene, expressing it in a plant and testing the ability of the expressed amidase to increase Acephate toxicity for an insect pest exposed to the plant and to the pro-pesticide.

Cloning and Characterisation of Drosophila Amidase Genes

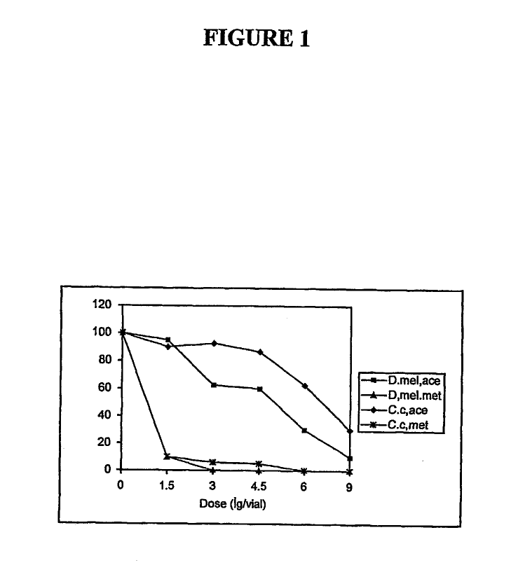

Drosophila melanogaster and Ceratitis capitata (medfly) flies are sensitive to Acephate and to Methamidophos (FIG. 1). Compared to Methamidophos, Acephate is less active. A search of the Drosophila Genome Database for amidases reveals six entries of putative amidase-like genes, based on sequence homology with known amidases. The relative database entries are:

1. Q9VHWO (FBgn0037547; CG7910, 530 AA)

2. Q9VHV9 (FBgn0037548; CG7900, 520 AA)

3. Q9VDL3 (FBgn0038803; CG5191, 408 AA)

4. Q917I6 (FBgn0038803; CG5191, 507 AA)

5. Q9VBQ5 (FBgn0039341; CG5112, 523 AA)

6. Q9V699 (FBgn0033717; CG8839, 529 AA)

Entries Q9VDL3 and Q9I7I6 correspond to the same gene, (FBgn0038803; CG5191) but differ in transcript structure.

Five pairs of oligonucleotide primers are designed for polymerase chain reaction (PCR). The primers are selected so that the amplified fragments contained regions of the genes that are evolutionarily conserved. The sequences of these primers are shown below.

| | | |
|---|---|---|
| CG5112: | Forward | 5′ CCATTATCATCGCCACCAG 3′ |
| | Reverse | 5′ GTACATAACCCGTTCGGTTTC 3′ |

Expected PCR product size 491 bp

| | | |
|---|---|---|
| CG5191: | Forward | 5′ ATTTCAATGCCAAGCGGG 3′ |
| | Reverse | 5′ CGATCATGCAGTTGTAACC 3′ |

Expected PCR product size 372 bp

| | | |
|---|---|---|
| CG7900: | Forward | 5′ 1CTCTATTCGGCATTGGCTC 3′ |
| | Reverse | 5′ TGCGTCGCATAAGTTCAAAG 3′ |

Expected PCR product size 597 bp

| | | |
|---|---|---|
| CG7910: | Forward | 5′`CGATGTGGTTGAACTGGTCC 3′ |
| | Reverse | 5′`ATCGCCCGCCATTATTTCC 3′ |

Expected PCR product size 403 bp

| | | |
|---|---|---|
| CG8839: | Forward | 5′ GGAGGAGTTGGAGAAGGAGAAG 3′ |
| | Reverse | 5′ TCTCTCTCAAGTGCTGTGCC 3′ |

Expected PCR product size 810 bp

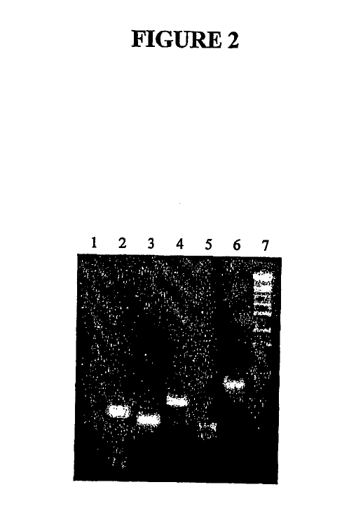

PCR is performed using the above primers and Drosophila genomic DNA as template (FIG. 2). The amplified DNA fragments are of the expected size and are shown by sequence analysis to correspond to the target genes.

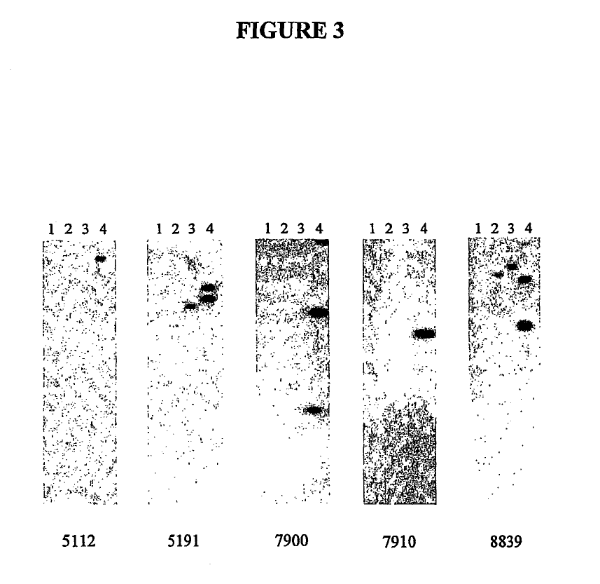

The PCR fragments are used to identify amidase genes in Drosophila and other dipteran species by Southern blot analysis (FIG. 3). Genomic DNA from four dipteran species, Drosophila melanogaster (family Drosophilidae), Ceratitis capitata and Bactrocera oleae (family Tephritidae) and Musca domestica (family Muscidae) is digested with EcoRI and hybridised with the 32P-labelled PCR fragments. All probes give strong hybridisation signal with Drosophila, as expected. Two of the genes (CG5191 and CG0839) give weaker signal with the two Tephritid fruit flies (medfly and olive fly). No signal is detected in the more distantly related house fly with any of the probes used. These results demonstrate that all probes can be used to identify the corresponding Drosophila genes and that the CG5191 and CG8839 probes are useful for identifying the corresponding homologous genes from medfly and olive fly. The results also strongly suggest that the CG5191 and CG8839 probes may be useful for identifying amidase genes from other diptera belonging to the families Drosophilidae and Muscidae.

All five Drosophila amidase genes contain introns, which are likely to remain unprocessed, or to be inappropriately spliced in plant cells. To avoid this, amidase cDNA sequences are cloned for expression in plants. The five genes from Drosophila are cloned by RT-PCR amplification with primers designed to cover the two ends of the coding region (from the initiating ATG to the terminating codon). The primers contain restriction sites to facilitate directional cloning of the gene into expression vectors. Poly(A)+RNA is used as template for RT-PCR The RNA is from the appropriate stage and/or tissue of Drosophila, as determined by previous Northern analysis.

To determine which amongst the amidase genes are capable of catalysing the Acephate to Methamidophos conversion, the genes are introduced into a Drosophila expression cassette containing the Drosophila heat-inducible hsp70 promoter which is introduced into the genome of D. melanogaster by Minos transposon-mediated germ line transformation. Transformed lines are established and tested for sensitivity to Acephate with and without previous heat-shock treatment. Lines expressing the converting amidase show increased sensitivity to the pro-insecticide.

The same expression vectors are used for transient expression experiments in a Drosophila cell line, as described in the literature. In these experiments, plasmid DNA is transfected into cultured cells by calcium co-precipitation, and the transfected cells are assayed for the presence of the gene product 1-2 days post-transfection. The assay in this example is based on direct detection of Acephate to Methamidophos conversion, which is performed by standard analytical Gas Chromatography/Mass Spectrometry.

### Example 2

### Cloning and Characterisation of a Bacterial Amidase Gene

The steps involved in this procedure include (a) screening a collection of bacterial species to identify species that convert Acephate to Methamidophos, and b) cloning the responsible amidase by complementation in a bacterium that does not convert If the genome sequence of the converting bacterium is available, it is possible to identify candidate amidases by homology to other known amidases, of prokaryotic or eukaryotic origin.

Transgenic Nicotiana tabacum is generated by standard procedures, expressing a converting enzyme in the leaves. Expression cassettes for this purpose have been described in the literature (Christov N K, Imaishi H, Ohkawa H. (1999) Green-tissue-specific expression of a reconstructed crylC gene encoding the active fragment of bacillus thuringiensis delta-endotoxin in haploid tobacco plants conferring resistance to Spodoptera litura. Biosci Biotechnol Biochem 63:1433-1444). The effect of the transgene is assayed directly, by detection of Methamidophos in Acephate treated plants by Gas Chromatography/Mass Spectrometry.

An insect bioassay is used to assess efficacy of the method. A lepidopteran “chewing pest”, such as the cotton bollworm Heliothis armigera, or an aphid “sucking pest”, such as the peach aphid (Myzus persicae) are used in this assay. Transgenic and non-transgenic control plants grown in separate environmental chambers are sprayed with Acephate, the insects are introduced in the chamber and insect survival is followed for a period of 24 hours. Methamidophos is used as a positive control.

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

We claim:

1. A method for controlling a population of target pests, comprising:
(i) providing a gene comprising a coding sequence encoding one constituent of an enzyme/pro-insecticide system and a regulatory region operatively limited to the coding sequence in the target plant;
(ii) transforming the population of target plants with the gene, and;
(iii) administering to the transformed population of target plants the remaining constituent(s) of the enzyme/pro-insecticide system, such that inactive pro-insecticide is converted to insecticide within the transformed plant;
wherein the coding sequence encodes an amidase enzyme, mixed functional oxidase/cytochrome P450 or an esterase enzyme which converts a pro-insecticide into its active metabolite.

2. A method according to claim 1, wherein the regulatory region is active to express the coding sequence predominately in plants.

3. A method according to claim 1 wherein the pro-insecticide comprises an organophosphate, phosphoramidate, neonicotinoid, or oxadaizine derivative.

4. A method according to claim 1 wherein the pro-insecticide/activation enzyme system comprises:
(i) acephate or an analogue of acephate; and
(ii) an amidase capable of converting acephate or an analogue of acephate to its active metabolite methamidophos.

5. A method according to claim 1 wherein the pro-insecticide/activation enzyme system comprises:
(i) N-Me-imidacloprid or an analogue of N-Me-imidacloprid; and
(ii) a mixed functional oxidase/cytochrome P450 capable of converting N-Me-imidacloprid or an analogue of N-Me-imidacloprid to its active metabolite imidacloprid.

6. A method according to claim 1, wherein pro-insecticide/activation enzyme system comprises:
(i) DPX-JW062 or an analogue of DPX-JW062; and
(ii) an esterase capable of converting DPX-JW062 or an analogue of DPX-JW062 to its active metabolite.

7. A method according to claim 1 where the pro-insecticide or conversion to its active insecticide in the host organism can be used to control plant pathogens including insects, nematodes, protozoa, fungi, bacteria and viruses.

8. A method according to claim 1 for the control of sucking, biting and chewing insects.

9. A vector which is capable of transforming a target plant cell, which vector comprises a gene comprising a coding sequence encoding one constituent of an enzyme/pro-insecticide system and a regulatory region operatively linked to the coding sequence in target plant, wherein the coding sequence encodes an amidase enzyme, mixed functional oxidase/cytochrome P450 or an esterase enzyme which converts the pro-insecticide into its active metabolite.
